(19) United States
(12) Patent Application Publication
(10) Pub. No.: US 2026/0298783 A1
(43) Pub. Date: Oct. 1, 2026

(54) CHARGED HYDROGELS FOR CONCENTRATING PROTEINS

(71) Applicant: MASSACHUSETTS INSTITUTE OF TECHNOLOGY, Cambridge, MA (US)

(72) Inventors: Kripa K. Varanasi, Lexington, MA (US); Baptiste Blanc, Somerville, MA (US); Fabian Dickhardt, Cambridge, MA (US); Bert Joannes C. Vandereydt, Boston, MA (US)

(73) Assignee: MASSACHUSETTS INSTITUTE OF TECHNOLOGY, Cambridge, MA (US)

(21) Appl. No.: 19/479,724

(22) PCT Filed: Apr. 29, 2024
(86) PCT No.: PCT/US2024/026903

§ 371 Date: Oct. 28, 2025

Related U.S. Application Data
(60) Provisional application No. 63/499,229, filed on Apr. 29, 2023.

(51) Int. Cl. G01N 1/40

(52) U.S. Cl. CPC G01N 1/405

(57) ABSTRACT

Hydrogel compositions that are capable of separating analytes in a mixture of analytes, and related methods, are generally described. For example, a hydrogel composition can be mixed with a sample comprising, at least, a first analyte and a second analyte, and the hydrogel composition can partition the first analyte selectively over the second electrolyte. The first analyte remains confined by the hydrogel composition, while the second analyte remains unconfined, facilitating separation of the two analytes.

### RELATED APPLICATIONS

This application claims priority under 35 U.S.C. § 119(e) to U.S. Provisional Application No. 63/499,229, filed Apr. 29, 2023, and entitled “CHARGED HYDROGELS FOR CONCENTRATING PROTEINS,” which is incorporated herein by reference in its entirety for all purposes.

### TECHNICAL FIELD

Hydrogel compositions that are capable of separating analytes in a mixture of analytes, and related methods, are generally described.

### BACKGROUND

Concentrating proteins is an important issue in numerous fields and has many industrial applications, from the functioning of eukaryotic cells containing various organelles that harbor distinct chemical microenvironments, to protein separation, drug delivery and soft actuators. Protein purification is conventionally achieved by extracting a protein of interest from a crude (i.e., unpurified) extract, followed by iterations of centrifugation and fractionation. After each iteration, fractions believed to include the protein of interest undergo additional iterations of centrifugation and fractionation, while fractions believed to be free of the protein of interest are discarded. Of course, other methods of protein separation are known. For example, dialysis can also be used separate proteins. Dialysis involves placing the crude protein extract in a bag made of a selectively permeable membrane and exchanging the solvent used to make the crude protein extract. The bag is suspended in a larger volume comprising a buffered solution and the selectively permeable membrane is selected to be permeable to water, the buffer, and other components, but not to the protein of interest. Accordingly, water, buffers, and the other components can exchange between the bag and the larger volume until an equilibrium is established between the bag and the outer volume and protein of interest can be separated from proteins that can diffuse out of the bag and into the larger volume. Other methods of protein separation or purification are known.

While there exist several techniques to separate and purify proteins, these techniques suffer from some significant drawbacks. For example, centrifugation and fractionation typically requires the addition of a protein denaturant, such as a detergent, in order to separate proteins within the crude protein extract from one another. The inclusion of the protein denaturant(s) may contaminant the crude protein extract or otherwise interfere with the protein of interest. In some cases, significant trial and error is required to select the appropriate protein denaturant(s) to isolate the protein of interest from other species, and, in some cases still, it may not be possible to isolate a protein of interest even with the desired protein denaturants. Moreover, dialysis requires large volumes (e.g., to allow ions/salts/buffers to diffuse in and out from the bag to the larger volume), sophisticated membranes, and external equipment for flowing buffers to and from the larger volume.

Accordingly, improved articles and methods are desired to improve protein purification, and the purification of other biomolecules.

### SUMMARY

The following disclosure describes articles, systems, and methods related to separating or purifying a biomolecule from other species. The subject matter of the present disclosure involves, in some cases, interrelated products, alternative solutions to a particular problem, and/or a plurality of different uses of one or more systems and/or articles.

In one aspect, an article configured to sequester a charged analyte is described, the article comprising a porous structure comprising a polymeric surface, wherein greater than or equal to 25% of pores of the porous structure are of a size through which an analyte having a molecular weight of greater than or equal to 30 kDa may pass; a plurality of sites of charge associated with the polymeric surface; and wherein the analyte has a charge opposite the plurality of sites of charge associated with the polymeric surface.

In another aspect a system is described, the system comprising a first phase comprising a solution; a second phase comprising a porous polymeric material; an analyte; and one or more species; wherein a concentration of the analyte is higher in the second phase relative to the first phase, and wherein a concentration of the one or more species is higher in the first phase relative to the second phase.

In another aspect, a system is described, the system comprising a solution; a porous structure comprising a polymeric surface; an analyte having a molecular weight of greater than or equal to 30 kDa; and one or more species having a molecular weight of less than or equal to 20 kDa; wherein the analyte and the one or more species have the same charge.

In another aspect, a method for preparing a biological sample for determination of an analyte, the method comprising exposing the biological sample to a porous structure having a hydrophilic surface comprising a plurality of sites of charge associated with the hydrophilic surface, wherein at least 25% of the pores of the porous structure are of a size into which an auxiliary biomolecule in the sample can pass, wherein the auxiliary biomolecule has charge opposite the sites of charge of the hydrophilic surface of the porous structure; allowing at least some of the auxiliary biomolecule to be sequestered by the porous structure; and at least partially separating the sample from the porous structure, thereby providing the sample with an analyte concentration greater, relative to that of the auxiliary biomolecule, than the analyte/auxiliary biomolecule ratio in the sample prior to exposing.

In yet another aspect, a method for at least partially separating species in a sample comprising a first species and a second species, the method comprising mixing a hydrogel composition with the sample; partitioning the first species into the hydrogel at a higher concentration than the second species; and removing the hydrogel composition from the sample.

Another aspect of the disclosure herein is a method of enriching analytes in plasma, the method comprising preparing a liquid plasma sample; adding a charged polymeric hydrogel to the sample to produce a mixture; incubating the mixture for 1 to 1,000 minutes; separating the liquid sample from the charged hydrogel, wherein the hydrogel absorbs charged proteins from the sample. In some aspects of the disclosed method, the charged hydrogel comprises a polymer comprising a backbone, wherein fixed charges bound to the backbone. In some aspects embodiment of the disclosed method, the charged hydrogel comprises 2-aminoethyl methacrylamide hydrochloride. In some aspects of the disclosed method, the mass of the hydrogel is between 0.09 and 0.54 g. In some aspects of the disclosed method, the method is performed at pH 7.4. In some aspects of the disclosed method, the charged proteins comprise albumin, globulins, and/or fibrinogen. In some aspects of the disclosed method, the method is a high-throughput method using 96-well plates, preferentially robotically. Other aspects of the disclosed method are described elsewhere herein.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C show (a) a schematic of the concept to remove selectively the excess of albumin into a blood plasma sample with the steps 1) 2) 3) of the schematic represents a schematic of the experimental protocol used in the majority of experiments. Hydrogels of various masses, sizes and with tunable molecular properties are added to 1 mL of biomolecules solution. The biomolecules solution contains proteins and small molecules of various charges and sizes. (b) The concentration of the biomolecules in solution is measured over time using absorbance experiments at 280 nm. (c) The partition coefficient which is the ratio of concentration of the biomolecules in the hydrogel and in the solution at thermodynamic equilibrium is used to characterize the absorption capacity of the system, according to some embodiments;

FIGS. 2A-2E show (a) albumin (BSA) concentration in solution as a function of time for solutions of 1 mL of BSA at 40 mg·mL⁻¹. The mass of hydrogel added to the solution is varied and depicted in a blue color scale. The higher the hydrogel mass is, the darker the color is. (b) Partition coefficient of albumin as a function of the mass of the hydrogel. (c) Partition coefficient of albumin as a function of the initial concentration of albumin in the solution. (d) Mass of albumin captured in the hydrogel for a solution with an initial concentration of albumin of 40 mg·mL⁻¹ as a function of the hydrogel mass added. (e) Timescale of absorption of the albumin in the hydrogel as a function of the hydrogel thickness. The curves of albumin concentration vs time are fitted with a functional form

$$c ( t ) = ( {c}_{0} - {c}_{s} ) exp ( - \frac{t}{τ} ) + {c}_{s} ,$$

where c₀, cₛ, τ correspond respectively to the initial concentration of albumin, the steady concentration of albumin and the timescale of the absorption process, according to some embodiments;

FIGS. 3A-3B describes (a) A systematic studies of partition coefficient as a function of the amount of charge of the polymer backbone of the hydrogel for a solution of 40 mg·mL⁻¹ of BSA. X is the molar fraction of the charged monomer, 1-X is the fraction of the non-charged monomer, the acrylamide monomer. (b) Partition coefficient of BSA as a function of hydrogel mass for different salinity of the solution, according to some embodiments; and

FIGS. 4A-4B shows (a) Partition coefficient for various biomolecules as a function of the charge of the protein for solution at 1 mg·mL⁻¹, with [NaCl]=0.137M at pH=7.4 (b) Picture of the dyed albumin at equilibrium showing visually that BSA goes preferentially into the hydrogel, according to some embodiments.

### DETAILED DESCRIPTION

The following disclosure describes articles, systems, and methods for separating or purifying a biomolecule from other species (e.g., salts, small molecules, other biomolecules). In light of the drawbacks and limitations of existing protein separation and purification techniques, the present disclosure describes porous structures with polymeric surfaces, such a hydrogel, that may be added to suspension or a solution comprising the biomolecule of interest (e.g., an analyte) and other species (e.g., salts, small molecules, other biomolecules). The porous structure comprising the polymeric surface can selectively bind to the biomolecule of interest while being less selective for the other species in the suspension or solution. As is described in more detail below, the porous structure comprising the polymeric surface can partition the biomolecule of interest from the other species, thereby separating the biomolecule from the other components and purifying the suspension or solution. In some cases, the porous structure comprising the polymeric surface also includes a magnetic material (e.g., magnetic nanoparticles) to facilitate removal from the purified suspension or solution. Of course, the articles, systems, and methods described herein may be used in other direction, that is, to remove a biomolecule from the other species, where the other species comprise another biomolecule of interest, and it is desired to remove the first biomolecule from the other biomolecules. Other variations and combinations of the articles, system, and methods are described below.

Various of the embodiments described herein include a porous structure, for example, a porous structure comprising a polymeric surface. For example, in some embodiment, the article is or comprises a porous structure comprising a polymeric surface. In some embodiments, the porous structure comprising the polymeric surface comprises a hydrogel. In part due to their hydrophilicity, hydrogels may be particularly useful in the context of biological systems or other aqueous-based systems.

As noted above, a porous structure may comprise a polymeric surface. The polymeric surface may comprise a variety of polymeric materials. For example, in some embodiments, the polymeric surface comprises a polyacrylamide (e.g., poly(acrylamide)-gum acacia). Additional non-limiting examples of suitable polymeric materials include chitosane, hyaluronic acid, methacryloyl-L-lysine, N-[3-(N, N-dimethylamino) propyl] methacrylamide, N-[2-(N, N-dimethylamino)ethyl] methacrylamide, N-[2-(N, N-dimethylamino)ethyl] methacrylamide, 2-(N, N-dimethylamino)ethyl acrylate, 2-(N, N-diethylamino)ethyl methacrylate, 2-acryloxyethyltrimethylammonium chloride, isopropylacrylamide, 3-sulfopropyl methacrylate potassium salt, and/or 2-sulfoethyl methacrylate. Other polymeric materials are possible as this disclosure is not so limited.

The polymeric surface (e.g., of a porous structure, of a hydrogel) as described herein may include a plurality of sites or areas of charge, which can impact the ability of the porous structure to sequester an analyte (e.g., a biomolecule). For example, the polymeric surface may be or comprise a polymeric “backbone,” with charged regions or moieties. Those skilled in the art, in view of the present disclosure, will understand that charge of the polymeric surface can be modified or modulated, as desired. For example, the monomeric components of the polymeric surface may be selected such that the polymeric surface has an excess of positive or negative charge. In some embodiments, the sites of charge are selected in view of the analyte (e.g., a biomolecule of interest). For example, in some embodiments, the analyte is suspected of being an anion, and the plurality of sites of charge are positively charged. In some embodiments, the analyte is suspected of being a cation, and the plurality of sites are negatively charged.

As mentioned above, many embodiments include a porous structure (e.g., a hydrogel). For example, in some embodiments, an average pore size of the pores is greater than or equal to 0.1 μm, greater than or equal to 0.2 μm, greater than or equal to 0.5 μm, greater than or equal to 0.7 μm, greater than or equal to 1 μm, greater than or equal to 2 μm, greater than or equal to 3 μm, or greater than or equal to 5 μm. In some embodiments, an average pore size of the pores is less than or equal to 5 μm, less than or equal to 3 μm, less than or equal to 2 μm, less than or equal to 1 μm, less than or equal to 0.7 μm, less than or equal to 0.5 μm, less than or equal to 0.2 μm, or less than or equal to 0.1 μm. Combinations of the above-referenced ranges are also possible (e.g., greater than or equal to 0.1 μm and less than or equal to 5 μm). Other ranges are possible.

The volumetric porosity of the porous structure may be within any suitable range or value. In some embodiments, the porosity of the porous structure is at least 20%, at least 30%, at least 40%, at least 50%, at least 60%, or at least 70%. In some embodiments, the porosity of the porosity of the porous structure is less than or equal to 70%, less than or equal to 60%, less than or equal to 50%, less than or equal to 40%, less than or equal to 30%, or less than or equal to 20%. Combinations of the foregoing ranges are possible (e.g., at least 20% and less than or equal to 70%). Other ranges are possible.

In some embodiments, the porous structure has a particular thickness (e.g., before adding the porous structure to a suspension or solution). In some embodiments, a thickness of the porous structure is greater than or equal to 0.1 mm, greater than or equal to 0.2 mm, greater than or equal to 0.5 mm, greater than or equal to 0.7 mm, greater than or equal to 1 mm, greater than or equal to 1.3 mm, greater than or equal to 1.5 mm, greater than or equal to 1.7 mm, greater than or equal to 2 mm, greater than or equal to 2.5 mm, or greater than or equal to 3 mm. In some embodiments, a thickness of the porous structure is less than or equal to 3 mm, less than or equal to 2.5 mm, less than or equal to 2 mm, less than or equal to 1.7 mm, less than or equal to 1.5 mm, less than or equal to 1.3 mm, less than or equal to 1 mm, less than or equal to 0.7 mm, less than or equal to 0.5 mm, less than or equal to 0.2 mm, or less than or equal to 0.1 mm. Combinations of the above-referenced range are also possible (e.g., greater than or equal to 0.1 mm and less than or equal to 3 mm). Other ranges are possible.

In some embodiments, a polymeric precursor (e.g., monomers, initiators) is used to form the porous structure in situ within the sample.

In some embodiments, the porous structure (and/or the article itself) is transparent. That is to say, in some embodiments, the porous structure permits at least a first wavelength to pass through with little to no reflection or deflection.

In some embodiments, the porous structure may also include a magnetic material, which may facilitate removing the porous structure (e.g., along with a biomolecule of interest) from a sample (e.g., after it has sequestered the biomolecule of interest). Any suitable magnetic material can be used. For example, in some embodiments, the porous structure also comprises magnetic nanoparticles, and the nanoparticles can be formed while the porous structure is forming, or provided to the porous structure after forming the porous structure.

As noted above and elsewhere herein, porous structures may comprise magnetic particle (e.g., magnetic nanoparticles). In some embodiments, an average size of the magnetic particles is greater than or equal to 1 nm, greater than or equal to 10 nm, greater than or equal to 20 nm, greater than or equal to 30 nm, greater than or equal to 50 nm, or greater than or equal to 100 nm. In some embodiments, an average size of the magnetic particles is less than or equal to 100 nm, less than or equal to 50 nm, less than or equal to 20 nm, less than or equal to 10 nm, or less than or equal to nm. Combinations of the above-referenced ranges are also possible (e.g., greater than or equal to 1 nm and less than or equal to 100 nm). Other ranges are possible as this disclosure is not so limiting.

As mentioned elsewhere herein, the article, systems, and methods described herein may be used to separate a biomolecule from other species, including other biomolecules. The biomolecule of interest may be designated as the analyte, while other species may be present. This disclosure is not limited by the type or kind of biomolecule, and those skilled in the art would understand that a biomolecule encompasses many types of species, including peptides, proteins, polysaccharides, nucleic acid, and so forth. In one embodiment, the biomolecule of interest is albumin. Of course, other biomolecules are possible. Additional non-limiting example include troponin, protein tau, hemoglobin, fibrinogen, and/or cytokines.

A biomolecule of interest (e.g., an analyte) may be of a size as to pass through one or more pores of the porous structure. That is to say, the porous structure may comprise a plurality of pores sized to permit the biomolecule of interest to pass into a volume defined by the porous structure. In some embodiment, the porous structure may permit a biomolecule having a molecular weight of greater than or equal to 30 kDa, greater than or equal to 50 kDa, greater than or equal to 70 kDa, greater than or equal to 100 kDa, greater than or equal to 150 kDa, greater than or equal to 200 kDa, greater than or equal to 250 kDa, greater than or equal to 300 kDa, greater than or equal to 400 kDa, greater than or equal to 500 kDa, or greater than or equal to 1,000 kDa. In some embodiments, the porous structure may permit a biomolecule having a molecular weight of less than or equal to 1,000 kDa, less than or equal to 500 kDa, less than or equal to 400 kDa, less than or equal to 300 kDa, less than or equal to 250 kDa, less than or equal to 200 kDa, less than or equal to 150 kDa, less than or equal to 100 kDa, less than or equal to 70 kDa, less than or equal to 50 kDa, or less than or equal to 30 kDa. Combinations of the above-referenced ranges are also possible (e.g., greater than or equal to 30 kDa and less than or equal to 50 kDa). Other ranges are possible.

As mentioned in relation to the plurality of sites of charge elsewhere herein, the biomolecule may have the same or different charge as the plurality of sites of charge. For example, in some embodiments, the plurality of sites of charge are negative, and the biomolecule is (or is suspected of being) negatively charged. In some embodiments, the plurality of sites of charge are negative, and the biomolecule is (or is suspected of being) positively charge. While charge is one component of the porous structure to consider, a biomolecule may be sequestered (or rejected), at least in part, based on other parameters (e.g., porosity, pore size, ion screening) as described elsewhere herein.

The articles, systems, and methods described herein are useful in separating a biomolecule (e.g., a protein), or some other component, from other species (e.g., a second species, a third species, lysozyme, lactate) within a suspension or a solution. For example, a sample can be obtained from a subject (e.g., a patient, a human, an animal, a plant), or some other source (e.g., a cell culture), and mixed with a solvent (e.g., water, buffer solution) to form a solution or suspension comprising (or suspected of comprising) the biomolecule of interest, along with other species to be separated from the protein of interest. In some embodiments, the porous structure (e.g., a hydrogel) is added to the sample, and the porous structure can sequester the protein of interest with a greater affinity for the protein of interest relative to the other species in the sample. As described elsewhere herein, properties of the porous structure and/or the biomolecule (e.g. size, charge), at least in part, affect the retention of the biomolecule of interest.

In some embodiments, the sample (e.g., a solution or a suspension) may have two or more phases, for example, a first phase and a second phase. By way of illustration, the sample may comprise a variety of components (e.g., a biomolecule of interest, other biomolecules not of interest, salts, small molecules, etc.), some of which may or may not be dissolved in the solvent. In some embodiments, the porous structure in the sample comprises a phase of the sample. In some such embodiments, the solvent (e.g., water) of the sample comprises the first phase, and the porous structure comprises the second phase. In some embodiments, the biomolecule of interest (e.g., the analyte) has a higher affinity for the second phase (e.g., the porous structure) relative to the first phase (e.g., the solvent of the sample or another phase of the sample).

In some embodiments, an affinity for one phase (e.g., a second phase, a porous structure within the sample) over another phase (e.g., a first phase, a solvent of the first phase) can be measured by determining a partition coefficient, as described in more detail elsewhere herein.

The pH of a sample can be adjusted in order to alter a charge state of a species (e.g., a biomolecule of interest, other species) in the sample. In some embodiments, a pH of the sample is (or is adjusted to) greater than or equal to 6.0, greater than or equal to 6.5, greater than or equal to 7.0, greater than or equal to 7.5, or greater than or equal to 8.0. In some embodiments, a pH of a sample is (or is adjusted to) less than or equal to 8.0, less than or equal to 7.5, less than or equal to 7.0, less than or equal to 6.5, or less than or equal to 6.0. Combinations of the foregoing ranges are also possible (e.g., greater than or equal to 6 and less than or equal to 8). Other ranges are possible as this disclosure is not so limiting.

In some embodiments, a partition coefficient for a biomolecule of interest is relatively high (e.g., greater than 1). In some embodiments, the partition coefficient for the biomolecule is greater than or equal to 2, greater than or equal to 3, greater than or equal to 5, greater than or equal to 10, greater than or equal to 20, greater than or equal to 30, greater than or equal to 50, greater than or equal to 70, or greater than or equal to 100. In some embodiments, the partition coefficient for the biomolecule is less than or equal to 100, less than or equal to 70, less than or equal to 50, less than or equal to 30, less than or equal to 20, less than or equal to 10, less than or equal to 5, less than or equal to 3, or less than or equal to 2. Combinations of the above-referenced ranges are also possible (e.g., greater than 1 and less or equal to 100). Other ranges are possible as this disclosure is not so limited.

In some embodiments, a partition coefficient for one or more other species in the sample is relatively low (less than 1). For example, if a first phase is the sample (or a solvent of the sample) and a second phase is the porous structure (e.g., a hydrogel), when the partition coefficient is less than 1, the other species have a higher affinity for the sample relative to the porous structure. In some embodiments, a partition coefficient for other species in the sample is less than or equal to 1, less than or equal to 0.8, less than or equal to 0.5, less than or equal to 0.3, less than or equal to 0.1, less than or equal to 0.05, less than or equal to 0.01, or less than or equal to 0.001. In some embodiments, a partition coefficient for the other species in the sample is greater than or equal to 0.001, greater than or equal to 0.01, greater than or equal 0.05, greater than or equal to 0.01, greater than or equal to 0.05, greater than or equal to 0.1, greater than or equal to 0.3, greater than or equal to 0.5, greater than or equal to 0.8, or greater than or equal to 1. Combinations of the foregoing ranges are also possible (e.g., greater than or equal to 0.001 and less than or equal to 1). Other ranges are possible, as this disclosure is not so limited.

For various embodiments, the porous structure (e.g., a hydrogel) may require time for a biomolecule of interest (e.g., an analyte) to partition within the porous structure (e.g., within pores of the porous structure). Accordingly, the porous structure may be exposed to the sample for a sufficient time for the biomolecule of interest to partition (e.g., diffuse) into the porous structure. In some embodiments, the porous structure is exposed to the sample for at least 1 minute, at least 5 minutes, at least 10 minutes, at least 20 minutes, at least 30 minutes, at least 60 minutes, at least 100 minutes, at least 250 minutes, at least 500 minutes, at least 750 minutes, or at least 1,000 minutes. In some embodiments, the porous structure is exposed to the sample for less than or equal to 1,000 minutes, less than or equal to 750 minutes, less than or equal to 500 minutes, less than or equal to 250 minutes, less than or equal 100 minutes, less than or equal to 60 minutes, less than or equal to 30 minutes, less than or equal to 20 minutes, less than or equal to 10 minutes, less than or equal to 5 minutes, or less than or equal to 1 minute. Combinations of the foregoing ranges are also possible (e.g., greater than or equal to 1 minute and less than or equal to 1,000 minutes). Other ranges are possible.

As described above and elsewhere herein, the articles, systems, and methods described herein may be useful for separating a biomolecule (e.g., the analyte) from other species. The other species are not particularly limited may include salts, buffers, ions, or other biomolecules.

In some embodiments, the other species (e.g., one or more species, a plurality of species) may be relatively small compared to the analyte. In some such embodiments, however, it will be understood that even if a particular species is small enough to pass through the pores of the porous structure, other parameters (e.g., charge, ion screening) may result in rejection of the species from the porous structure while the biomolecule of interest (e.g., the analyte) is retained by the porous structure. In some embodiments, the other species have a molecular weight of less than or equal to 20 kDa, less than or equal to 10 kDa, less than or equal to 1 kDa, less than or equal to 500 Da, less than or equal to 250 Da, less than or equal to 100 Da, less than or equal to 50 Da, less than or equal to 25 Da, less than or equal to 10 Da, less than or equal to 5 kDa, or less than or equal to 1 Da. In some embodiments, the other species have a molecular weight of greater than or equal to 1 Da, greater than or equal to 5 Da, greater than or equal to 10 Da, greater than or equal to 25 Da, greater than or equal to 50 Da, greater than or equal to 100 Da, greater than or equal to 250 Da, greater than or equal to 500 Da, greater than or equal to 1 kDa, greater than or equal to 10 kDa, or greater than or equal to 20 kDa. Combinations of the above-referenced ranges are also possible (e.g., less than or equal to 20 kDa and greater than or equal to 1 Da). Other ranges are possible.

In some embodiments, the sample includes other salts and/or buffers. In some embodiments, the salt and/or buffer is present as a concentration of at least 0.01 M, at least 0.05 M, at least 0.1 M, at least 0.3 M, at least 0.5 M, at least 0.7 M, or at least 1 M. In some embodiments, the salt and/or buffer is present at a concentration of less than or equal to 1 M, less than or equal to 0.7 M, less than or equal to 0.5 M, less than or equal to 0.3 M, less than or equal to 0.1 M, less than or equal to 0.05 M, or less than or equal to 0.01 M. Combinations of the foregoing range are also possible (e.g., at least 0.1 M and less than or equal to 1 M). Other ranges are possible.

The following examples are intended to illustrate certain embodiments of the present invention, but do not exemplify the full scope of the invention.

### Example 1

The present example describes work that involves the development of hydrogel magnetic nanocomposites for protein purification and heavy metal extraction applications. The magnetic nanoparticles (MNPs) were prepared in situ in poly(acrylamide)-gum acacia (PAM-GA) hydrogels. The formation of magnetic nanoparticles in the hydrogel networks was confirmed by Fourier transform infrared (FTIR) spectroscopy and X-ray diffraction (XRD). Scanning electron (SEM) microscopy studies revealed the formation of MNPs throughout the hydrogel networks. The average size of MNPs formed in the hydrogel networks was 3-5 nm as determined by transmission electron microscopy (TEM). The thermal properties of the hydrogel magnetic nanocomposites were evaluated by dynamic scanning calorimetry (DSC) and thermogravimetric (TG) analysis. The magnetic properties of the developed hydrogel magnetic nanocomposites were determined by a vibrating sample magnetometer (VSM). The swelling properties of the hydrogel and the hydrogel magnetic nanocomposites were studied in detail. The hydrogel magnetic nanocomposites are utilized for the removal of toxic metal ions such as Co(II), Ni(II), and Cu(II) and for protein purification. The results confirm that the hydrogel magnetic nanocomposites exhibit superior extraction properties to hydrogels.

Below, it is shown that the concentration of biomolecules can be modulated by using charged hydrogels. Hydrogels with controllable mesh sizes, charges and geometrical sizes are synthesized and added to aqueous solutions containing the biomolecules. The hydrogels interact distinctly with each biomolecule, hence, change the concentration of the biomolecules in and out of the aqueous solution. In this example, albumin, lysozyme, and lactate are used as model biomolecules at pH 7.4 and various salinities. The choice of these model system is guided by an application, related to biomarkers analysis in blood plasma where albumin concentration can be 1 billion time bigger than the concentration of the biomarkers of interest for biomedical inquiries. This large discrepancy in biomolecules concentration has hampered the analysis of blood plasma content.

To capture the physicochemical mechanisms involved in the partitioning of biomolecules, albumin was used as model biomolecule, a large negatively charged biomolecule with a molecular weight of 65 kDa, lysozyme, a large positively charged biomolecule with a molecular weight of 16 kDa and lactate, a small negatively charged biomolecule with a molecular weight of 90 Da. It is demonstrated that albumin, which is negatively charged partition strongly into the positively charged hydrogel, depleting the concentration of this biomolecule in the aqueous solution. In the contrary, lyzozyme, which is positively charged is not absorbed in the same hydrogel. Interestingly, a small negatively charged molecule such as lactate does not interact strongly with the hydrogel, and hence does not preferentially partition into it. By synthetizing hydrogels with various charges in the polymer backbone, it is shown that the charge of the hydrogel drives the absorption of albumin into the hydrogel. It is also shown that at high salinity, when the charges of the hydrogel can be screened with the counterions, the partitioning of albumin into the hydrogel drastically decreases. Taken together, these two results indicate that the electrostatic interaction of the hydrogel with the protein is one of the control parameter explaining its absorption. Finally, it is demonstrated that the timescale of absorption can be engineered by changing the size, here the thickness of the hydrogel. For albumin, it is shown that the effective diffusion coefficient is similar to the diffusion coefficient of albumin in solution, that was measured with light scattering.

### Results and Discussion

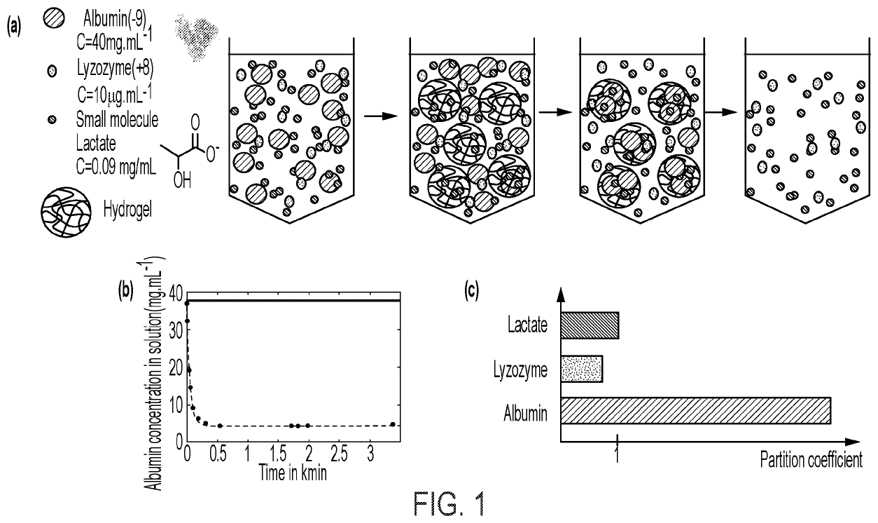

A schematic of the setup used to collect data is shown in FIG. 1A. Briefly, a solution of 1 mL is poured into a 2 mL microcentrifuge tube. A variable quantity of hydrogels with tunable properties and geometries are added to the solution. Over the entire course of the experiment, the tube is continuously stirred to homogenize the concentration of biomolecules in the solution. The concentration of biomolecules in the solution is measured using an absorbance measurement at 280 nm. With a micropipette, 2 μL is taken out of the solution and used to measure the concentration of the solution. From these measurements, the evolution of the concentration of the biomolecule in solution was followed as a function of time (FIG. 1B). After a transient time t, a steady state concentration value cₛ is eventually reached. By conservation of the moles of the biomolecules, the molar concentration of biomolecules in the hydrogel can be determined. To characterize further the steady state regime, the partition coefficient was computed, which is the ratio of the molar concentration of biomolecules in the hydrogel to the molar concentration of biomolecules in the solution. Hence, a high partition coefficient (e.g., greater than 1) indicates that the biomolecule preferentially goes in the hydrogel, whereas a low partition coefficient (e.g., less than 1) signifies that the biomolecule preferentially stays in the solution (FIG. 1C).

Since the model biomolecule is Bovine Serum Albumin, a demonstration of the approach began in the context of removing albumin from blood plasma samples, in which lower concentration is desired to measure the concentration of low abundance biomolecules. Indeed, many low-abundance biomarkers for early detection of cancer and other diseases are invisible to mass spectrometry because they exist in body fluids in very low concentrations and may be masked by high-abundance proteins such as albumin and immunoglobulins. Protein crashing is typically used to remove albumin and other large proteins from blood plasma with the constrain that it is not selective, and it can denature the biomolecules. The strategy described in this example consists in using electrostatic interaction to selectively capture BSA into a charged hydrogel. At pH 7.4, the pH of blood plasma, BSA is negatively charged, hence capturing BSA inside a positively charged hydrogel made of 2-aminoethylmethacrylamide hydrochloride was tested (FIG. 1B). It was observed that BSA is strongly absorbed by the hydrogel.

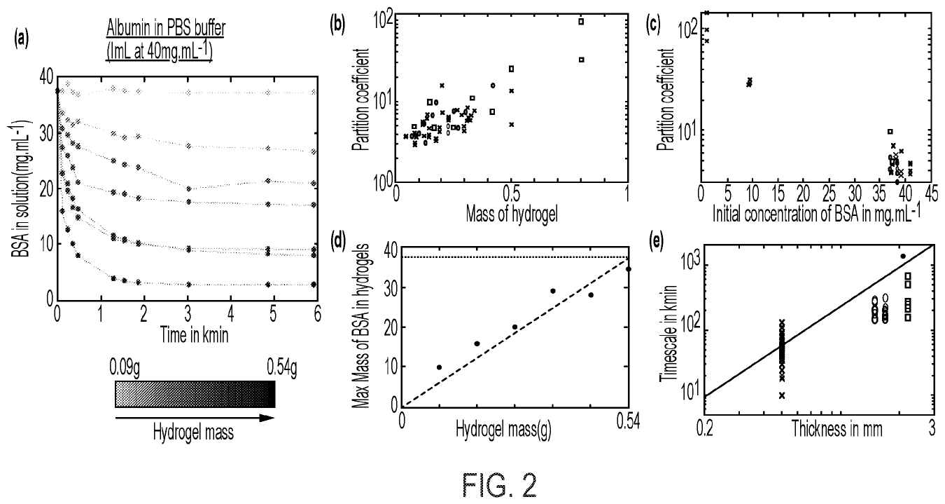

The effect of changing the mass of the hydrogel m, and thereby its volume, is shown in FIG. 2A. It was observed that the steady state concentration of BSA in the hydrogel increases linearly with its mass, m (FIG. 2D). The partition coefficient of BSA as a function of hydrogel mass and as a function of the initial concentration of BSA in solution was then computed. We report that for a given initial concentration of BSA of 40 mg·mL⁻¹, the more hydrogel there is, the larger the partition coefficient is (FIG. 2B). Interestingly, it was observed that the partition coefficient increases when the initial concentration of BSA decreases, given that the mass of the hydrogel is kept constant. These two experimental results spotlight an important role of the loading capacity of the hydrogel compared to the initial concentration of BSA.

The evolution of the concentration of BSA in the solution with time allowed for the extraction on a typical timescale to reach the steady state concentration value of BSA in solution. The experimental data is plotted with a functional form

$$c ( t ) = ( {c}_{0} - {c}_{s} ) exp ( - \frac{t}{τ} ) + {c}_{s} ,$$

where c₀, cₛ, τ correspond respectively to the initial concentration of albumin, the steady concentration of albumin and the timescale of the absorption process. Thin sheets experiments were then conducted of hydrogels with tunable thicknesses, h, and variable masses, m. In FIG. 2E, the timescale of the absorption process τ as a function of the thickness of the hydrogel h are shown. It was observed that the timescale of absorption varied with the square of the thickness of the hydrogel. The data was plotted with a functional form τ∝h². The proportionality coefficient matched the diffusion coefficient of albumin in solution. That the mass of the hydrogel does not drastically change the timescale τ was also verified.

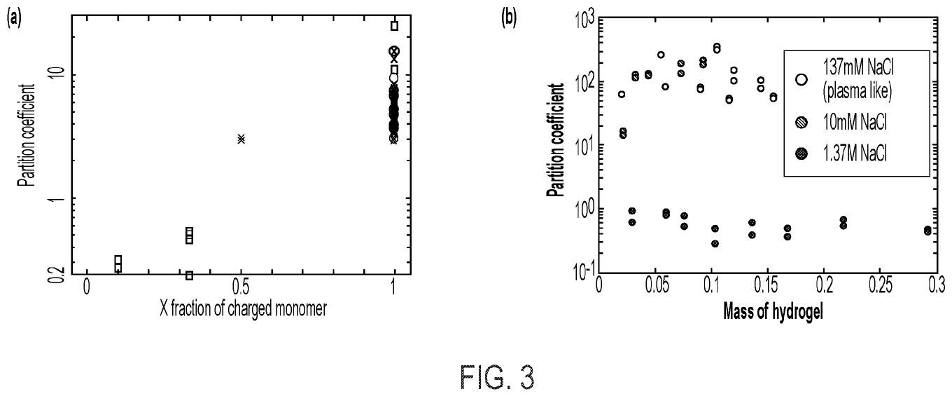

To understand the cause of the partition coefficient of BSA into a charged hydrogel, two sets of experiments were performed to test the effect of electric charges of the hydrogel on the partitioning of BSA. Hydrogels with tunable charges were synthesized by mixing the charged monomer 2-aminoethylmethacrylamide hydrochloride with the non-charged monomer, acrylamide. Rheological measurements and mass measurements allowed for the study of the effect of the charge of the hydrogel independently from the change of the hydrogel mesh size and the amount of polymer in it. It was observed that the charge of the hydrogel increased the partitioning of BSA into it, demonstrating that electrostatic interactions played an important role in the process. Since the phenomenon depends on the electrostatic interaction between the hydrogel and the biomolecules, it is expected that higher salt concentrations would screen the electrostatic interaction, reducing the partitioning of BSA into the charged hydrogel. To demonstrate this, experiments varying the salt concentration were conducted, e.g., by adding sodium chloride in the BSA solution. The size of the hydrogel is recorded as a function of the salinity to be sure that the partition measurement is not influenced by a change of the mesh size of the hydrogel. FIG. 3B shows that the partitioning of BSA decreases when the salinity of the solution increases, further validating that electrostatic interactions are critical for our phenomenon.

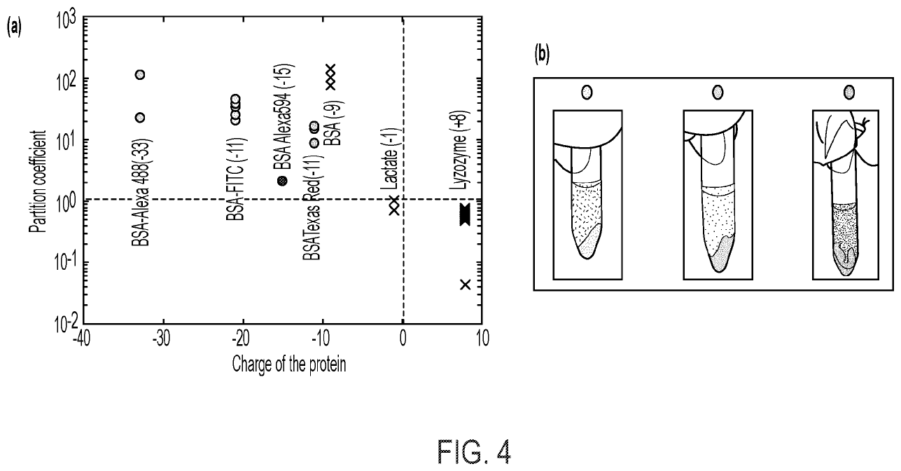

To show generalizability of the technique to other biomolecules, in FIG. 4A it is shown that tagging BSA with fluorophores, while still keeping the overall charge of BSA negative, does not change, qualitatively, its partition coefficient, but only change the magnitude of the partitioning. In comparison, a positively charged protein at pH 7.4, lysozyme, is shown to have a partition coefficient smaller than 1, meaning that this molecule preferentially does not go into the hydrogel. Therefore, a positively charged hydrogel allows separation of large biomolecules based on their charges even when the salinity of the solution is 0.137 M.

Interestingly, the size of the biomolecule plays also a role in its partitioning. To study the effect of the size of the analyte, lactate was chosen as one can measure its concentration in solution using a colorimetric assay. Lactate bears one negative charge at pH=7.4 and has a molecular weight of 90 Da. Lactate had a partition coefficient close to 1, meaning that lactate does not feel the presence of the polymer chain around it. This molecule is most certainly preferentially solvated by the water molecules and the sodium chloride salt which screens its negative charges, hence prevents its interaction with the charged polymer backbone. Taken together, these results show that hydrogels allow separation of biomolecules based on their charges and sizes.

### CONCLUSION

In this example, we demonstrate that hydrogels can modulate biomolecules absorption by using electrostatic interactions. The system works by controlling the charge of the polymer backbone, in comparison to the overall charge of the biomolecule. A positively charged hydrogel absorbs preferentially negatively charged biomolecules. The importance of the electrostatic effect is validated by varying the amount of charge in the polymer backbone constituting the hydrogel. Electrostatic effects are further established through experiments varying the salt concentration in the aqueous solution. Due to higher ionic screening of the electrostatic effect, the salt concentration decreases the absorption of the biomolecules in the charged hydrogel. Interestingly, the size of a biomolecule plays a role. A negatively charged molecule such as lactate is not sensitive to the charge of the hydrogels. Partitioning with hydrogels by size and electrostatic interaction can therefore be useful for concentrating biomolecules with applications in protein separation, drug delivery, or soft robotics. Extending biomolecules and mediums considered may provide valuable information on how different biomolecules types interact electrostatically with the charged hydrogels. Specifically, for the case of blood plasma analysis, where the current approach relies on protein crashing, this method has many advantages 1) the biomolecules are not denatured 2) the hydrogels can be washed and reused 3) the hydrogels does not dilute as much the protein content in the solution 4) the timescale of separation process can be engineered by changing the geometrical parameter of the hydrogels 5) the hydrogel can be integrated into a 96 well plate making the entire sample preparation automatable. With further development, nano-engineered porous hydrogels could be used for controlling absorption of biomolecules in blood plasma, allowing faster, more accurate measurement of biomarkers in blood plasma.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article configured to sequester a charged analyte, the article comprising:
a porous structure comprising a polymeric surface, wherein greater than or equal to 25% of pores of the porous structure are of a size through which an analyte having a molecular weight of greater than or equal to 30 kDa may pass;
a plurality of sites of charge associated with the polymeric surface; and
wherein the analyte has a charge opposite the plurality of sites of charge associated with the polymeric surface.

2. A system, comprising:
a first phase comprising a solution;
a second phase comprising a porous polymeric material;
an analyte; and
one or more species;
wherein a concentration of the analyte is higher in the second phase relative to the first phase, and
wherein a concentration of the one or more species is higher in the first phase relative to the second phase.

3. A system, comprising:
a solution;
a porous structure comprising a polymeric surface;
an analyte having a molecular weight of greater than or equal to 30 kDa; and
one or more species having a molecular weight of less than or equal to 20 kDa;
wherein the analyte and the one or more species have the same charge.

4. A method for preparing a biological sample for determination of a species, comprising:
exposing the biological sample to a porous structure having a hydrophilic surface comprising a plurality of sites of charge associated with the hydrophilic surface, wherein at least 25% of the pores of the porous structure are of a size into which an auxiliary biomolecule in the sample can pass, wherein the auxiliary biomolecule has charge opposite the sites of charge of the hydrophilic surface of the porous structure;
allowing at least some of the auxiliary biomolecule to be sequestered by the porous structure; and
at least partially separating the sample from the porous structure, thereby providing the sample with an analyte concentration greater, relative to that of the auxiliary biomolecule, than the analyte/auxiliary biomolecule ratio in the sample prior to exposing.

5. A method for at least partially separating species in a sample comprising a first species and a second species, the method comprising:
mixing a hydrogel composition with the sample;
partitioning the first species into the hydrogel at a higher concentration than the second species; and
removing the hydrogel composition from the sample.

6. The article, system, or method of claim 1, wherein the analyte is negatively charged.

7. The article, system, or method of claim 1, wherein the analyte is positively charged.

8. The article, system, or method of claim 1, wherein the porous structure comprises a hydrogel.

9. The article, system, or method of claim 1, wherein the polymeric surface comprises a surface of a hydrogel.

10. The article, system, or method of claim 4, wherein the biomolecule comprises albumin.

11. The article, system, or method of claim 1, wherein greater than or equal to 50% of pores of the porous structure are of a size through which an analyte having a molecular weight greater than 50 kDa may pass.

12. The article, system, or method of claim 1, wherein a molecular weight of the analyte is greater than or equal to 50 kDa.

13. The article, system, or method of claim 1, wherein a molecular weight of the analyte is less than or equal to 1,000 kDa.

14. The article, system, or method of claim 2, wherein a molecular weight of the one or more species is less than or equal to 1 kDa.

15. The article, system, or method of claim 2, wherein a molecular weight of the one or more species is greater than or equal to 20 Da.

16. The article, system, or method of claim 1, further comprising a magnetic material and wherein the magnetic material comprises magnetic nanoparticles.

17. The article, system, or method of claim 1, wherein the article is transparent.

18. The article, system, or method of claim 1, wherein the porous polymeric material comprises a hydrogel.

19. The article, system, or method of claim 1, wherein the porous polymeric material comprises a charged monomer and an uncharged monomer.

20. The article, system, or method of claim 5, wherein the hydrogel comprises a magnetic material and/or magnetic particles.

21-33. (canceled)
